(19) United States
(12) Patent Application Publication
(10) Pub. No.: US 2020/0306268 A1
(43) Pub. Date: Oct. 1, 2020

(54) VITAMIN COMPOSITION

(71) Applicant: Tempus Bio Technologies LLC, Cedar Hills, UT (US)

(72) Inventor: Tracy L. Holdford, Springfield, MO (US)

(73) Assignee: Tempus Bio Technologies LLC, Cedar Hills, UT (US)

(21) Appl. No.: 16/822,675

(22) Filed: Mar. 18, 2020

Related U.S. Application Data
(60) Provisional application No. 62/823,221, filed on Mar. 25, 2019.

(51) Int. Cl. A61K 31/593; A61K 9/00; A61K 9/107; A61K 47/26; A61K 47/28; A61K 47/14; A61K 47/24

(52) U.S. Cl. CPC A61K 31/593; A61K 9/006; A61K 9/1075; A61K 47/24; A61K 47/28; A61K 47/14; A61K 47/26

(57) ABSTRACT

A composition including nanoparticles encapsulating a vitamin, wherein the vitamin is surrounded by lipids for delivery through mucous membranes. A composition including nanoparticles encapsulating a vitamin, the nanoparticles including medium chain triglycerides, lecithin, cholesterol, and polysorbate 80. A pharmaceutical composition, including nanoparticles encapsulating a vitamin, the nanoparticles including medium chain triglycerides, lecithin, cholesterol, and polysorbate 80 in a pharmaceutically acceptable carrier. A method of making the composition, by forming nanoparticles encapsulating a vitamin, wherein the vitamin is surrounded by lipids for delivery through mucous membranes. A method of increasing absorption of a vitamin, by administering a composition including nanoparticles encapsulating a vitamin, wherein the vitamin is surrounded by lipids in a pharmaceutically acceptable carrier to a mucous membrane in an individual, the composition penetrating the mucous membrane and providing faster transit to the liver than other delivery systems, thereby increasing absorption of the vitamin.

### BACKGROUND OF THE INVENTION

### 1. Technical Field

The present invention relates to vitamin compositions. More specifically, the present invention relates to vitamin compositions with increased bioavailability.

### 2. Background Art

Vitamin D is a fat-soluble vitamin that is naturally present in very few foods, added to others, and available as a dietary supplement. It is also produced endogenously when ultraviolet rays from sunlight strike the skin and trigger vitamin D synthesis. Vitamin D obtained from sun exposure, food, and supplements is biologically inert and must undergo two hydroxylations in the body for activation. The first occurs in the liver and converts vitamin D to 25-hydroxyvitamin D [25(OH)D], also known as calcidiol. The second occurs primarily in the kidney and forms the physiologically active 1,25-dihydroxyvitamin D [1,25(OH)2D], also known as calcitriol. It has been known for years that absorption of vitamin D supplements through the stomach into the small intestines is not efficient. This results in large dosages of vitamin D taken for months yielding a very small increase in blood serum levels.

Certain individuals can also have decreased bioavailability of vitamin D, such as due to altered absorption in the small intestine or an altered metabolism in the individual's body. For example, obese individuals have been found to have decreased levels of vitamin D which may be due to body volume or sequestration of vitamin D in excess adipose tissue.

Very little research has been done in examining how to increase bioavailability of vitamin D and the effects of vehicles thereon. A comparison of studies by Grossman, et al. (Mol Nutr Food Res. 2010 August; 54(8): 1055-1061) showed that oil-soluble vehicles had the best bioavailability followed by powder-based vehicles, and then ethanol solutions.

There remains a need for a method of increasing vitamin D levels in the body effectively.

### SUMMARY OF THE INVENTION

The present invention provides for a composition for increasing absorption and bioavailability of a vitamin including nanoparticles encapsulating a vitamin, wherein the vitamin is surrounded by lipids for delivery through mucous membranes.

The present invention provides for a composition for increasing absorption and bioavailability of a vitamin including nanoparticles encapsulating a vitamin, wherein the nanoparticles include medium chain triglycerides, lecithin, cholesterol, and polysorbate 80.

The present invention also provides for a pharmaceutical composition for increasing absorption and bioavailability of a vitamin, including nanoparticles encapsulating a vitamin, wherein the nanoparticles include medium chain triglycerides, lecithin, cholesterol, and polysorbate 80, in a pharmaceutically acceptable carrier.

The present invention provides for a method of making the composition, by forming nanoparticles encapsulating a vitamin, wherein the vitamin is surrounded by lipids for delivery through mucous membranes.

The present invention provides for a method of increasing absorption of a vitamin, by administering a composition including nanoparticles encapsulating a vitamin surrounded by lipids in a pharmaceutically acceptable carrier to a mucous membrane in an individual, the composition penetrating the mucous membrane and providing faster transit to the liver than other delivery systems, thereby increasing absorption of the vitamin.

### DESCRIPTION OF THE DRAWINGS

Other advantages of the present invention are readily appreciated as the same becomes better understood by reference to the following detailed description when considered in connection with the accompanying drawings wherein:

FIG. 1 is a depiction of vitamin D3;

FIG. 2 is a table of medium chain triglycerides;

FIG. 3 is a depiction of phosphatidylcholine;

FIG. 4 is a depiction of cholesterol; and

FIG. 5 is a graph of vitamin D3 levels in subjects.

### DETAILED DESCRIPTION OF THE INVENTION

The present invention generally provides for nanoparticle compositions encapsulating vitamins that greatly increase the absorption and therefore bioavailability of the vitamin and greatly reduce the time to reach the liver for the first hydroxylation for activation. This is accomplished by delivery of the vitamin to mucous membranes anywhere in the body as further described below, which provides faster and more complete absorption of the vitamin than through the stomach as in the prior art. Most generally, the composition includes nanoparticles encapsulating a vitamin, wherein the vitamin is surrounded by lipids for delivery through mucous membranes.

“Mucous membrane” or “mucosa” as used herein, refers to any membrane that lines cavities of the body and covers surfaces of internal organs. It consists of one or more layers of epithelial cells overlying a layer of loose connective tissue. It is mostly of endodermal origin and is continuous with the skin at various body openings such as the eyes, ears, inside the nose, inside the mouth, lip, vagina, the urethral opening and the anus. Some mucous membranes secrete mucus, a thick protective fluid. The function of the membrane is to stop pathogens and dirt from entering the body and to prevent bodily tissues from becoming dehydrated.

The mucous membrane of organs is composed of one or more layers of epithelial cells that secrete mucus, and an underlying lamina propria of loose connective tissue. The type of cells and type of mucus secreted vary from organ to organ and each can differ along a given tract.

Mucous membranes line the digestive, respiratory, and reproductive tracts and are the primary barrier between the external world and the interior of the body; in an adult human the total surface area of the mucosa is about 400 square meters while the surface area of the skin is about 2 square meters. They are at several places contiguous with skin: at the nostrils, the lips of the mouth, the eyelids, the ears, the genital area, and the anus. Along with providing a physical barrier, they also contain key parts of the immune system and serve as the interface between the body proper and the microbiome. Some examples include: bronchial mucosa and the lining of vocal folds, endometrium: the mucosa of the uterus, esophageal mucosa, gastric mucosa, intestinal mucosa, nasal mucosa, olfactory mucosa, oral mucosa, penile mucosa, vaginal mucosa, frenulum of tongue, tongue, anal canal, and palpebral conjunctiva.

“Nanoparticle” as used herein, refers to a small particle generally between 1 and 100 nm in diameter. The preferred size of the nanoparticles of the present invention are described below.

“Lipids” as used herein, refers to a biomolecule that is soluble in nonpolar solvents (such as hydrocarbons).

The composition preferably includes a vitamin (and preferably vitamin D3 (cholecalciferol)), with the nanoparticles encapsulating the vitamin including the lipids of medium chain triglycerides, lecithin, and cholesterol, and polysorbate 80.

The vitamin used in the composition can be, but is not limited to, Vitamin A, Vitamin B₁, Vitamin B₂, Vitamin B₃, Vitamin B₅, Vitamin B₆, Vitamin B₁₂, Vitamin C, Vitamin D, Vitamin E, Vitamin K, and combinations thereof.

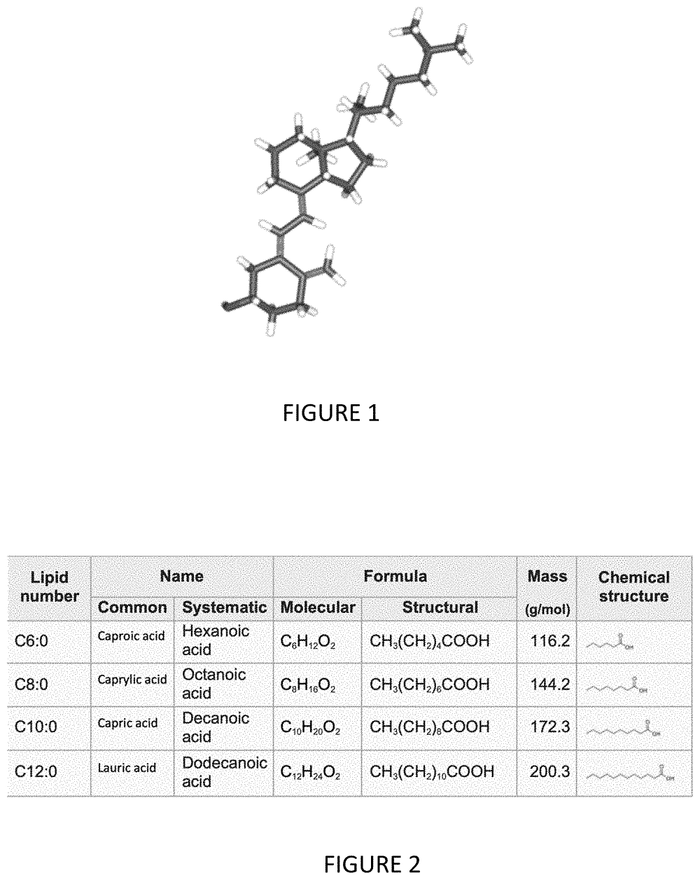

Most preferably, the vitamin is vitamin D3, shown in FIG. 1. Vitamin D3 generally has the following properties: formula C₂₇H₄₄O, molar mass of 384.64 g/mol, melting point of 181.4° F. (83° C.), boiling point of 925.6° F. (496.4° C.), ATC code A11CC05 (WHO), and IUPAC ID: (3β,5Z,7E)-9,10-secocholesta-5,7,10(19)-trien-3-ol. The vitamin D3 can be purchased from Sigma Aldrich as a research grade (100% purity by HPLC analysis).

Medium chain triglycerides (MCTs) are triglycerides with two or three fatty acids having an aliphatic tail of 6-12 carbon atoms, i.e., medium-chain fatty acids (MCFAs). Their hydroxyl value in 2.0 mg KOH/g is C6 0.0% (m/m), C8 59.2% (m/m), C10 40.6% (m/m), C12 0.2% (m/m), C14 0.0% (m/m). Additional properties of the MCTs are shown in FIG. 2. The MCTs can be in the form of oil, and this is used to change the vitamin from a powder to a liquid form. For example, vitamin D3 is not soluble in water but it is very soluble in MCT oil. Each of the types of MCTs described are present in the oil, and additional MCTs can also be present.

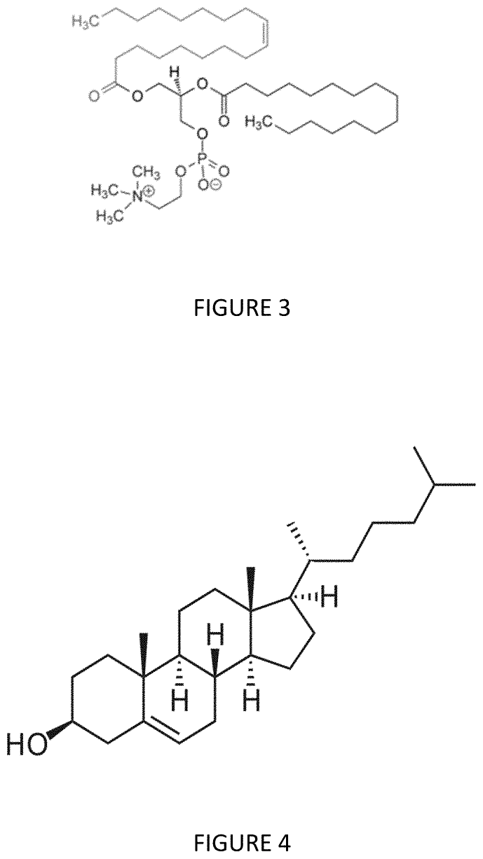

Lecithin is a generic term to designate any group of yellow-brownish fatty substances occurring in animal and plant tissues, which are amphiphilic, they attract both water and fatty substances. Lecithins are mixtures of glycerophospholipids including phosphatidylcholine, phosphatidylethanolamine, phosphatidylinositol, phosphatidylserine, and phosphatidic acid. An example of a phosphatidylcholine (a type of phospholipid in lecithin) is shown in FIG. 3, having a choline and phosphate group, glycerol, monosaturated fatty acid, and saturated fatty acid components. Choline and its metabolites are needed for several physiological purposes, including cell membrane signaling and cholinergic neurotransmission. Lecithin can be thought of as a chemical “key” and is used to gain access through the cells to the capillary system of the mouth.

Cholesterol, shown in FIG. 4, is a lipid with a unique structure consisting of four fused hydrocarbon rings forming the bulky steroid structure. There is a hydrocarbon tail linked to one end of the steroid and a hydroxyl group linked to the other end. The hydroxyl group is able to form hydrogen bonds with nearby carbonyl oxygen of phospholipid and sphingolipid head groups. Cholesterol is known as a “sterol” because it is made out of an alcohol and a steroid. Cholesterol is present in most animal membranes with varying amounts but is absent in prokaryotes and intracellular membranes. Cholesterol is also a key regulator of membrane fluidity in animals. It can insert itself into bilayers perpendicular to the membrane plane. The hydroxyl group forms hydrogen bonds with the carbonyl oxygen of a phospholipid head group while the hydrocarbon tail positions itself in the non-polar core of the bilayer. Since the structure of cholesterol differs from phospholipids, it disrupts the normal reactions between fatty acid chains. Cholesterol is also able to form lipid rafts when it forms specific complexes with certain phospholipids which results in membranes that are less fluid and less subject to phase transitions. This also increases the permeability of the cell membrane to hydrogen and sodium ions. Cholesterol is used to gain access through the cells to the capillary system of the mouth.

Polysorbate 80 (preferably TWEEN 80™ (Croda Americas, Inc.)) is a purified nonionic surfactant and emulsifier, produced by Sigma Aldrich and derived from polyethoxylated sorbitan and oleic acid. POE (20) sorbitan monooleate, polyethylene glycol sorbitan monooleate, and polyoxyethylenesorbitan monooleate are synonyms for TWEEN 80™ Polysorbate 80 is used as a lubricant to ensure that the mucus and the cells do not trap the vitamin within their structures.

The composition can further include FOODGARD® by BIOSECUR®, which is a natural antimicrobial that is added to the composition after nanosizing to retard any microbial growth.

An example of the formulation of the composition is as follows in TABLE 1.

TABLE 1

| | | |
|---|---|---|
| Vitamin D3 | 2 | grams |
| MCT oil | 4 | grams |
| Lecithin | 0.51688 | grams |
| Cholesterol | 0.0256 | grams |
| TWEEN 80 ™ | 1.4332 | grams |
| FOODGARD ® | 12 | grams |

The composition is formed in nanoparticles of 35-45 nm in size. Due to the nature of nanomaterials, is it preferable to keep the above amounts and ratios in order to keep the material stable in solution. However, other amounts can be used. The particular size of the nanoparticles is chosen to ensure that the vitamin molecule is intact and not fractured. If the vitamin breaks into its molecular components, they offer no health benefit.

The specific lipids in the formulation (MCT oil, lecithin, cholesterol) in combination with the size of the nanoparticles allow the cells of the mucous membrane to part enough to gain access to the capillary system, for example, that is present in great quantities in the mouth. This both decreases the time for the vitamin to reach the liver and eliminates and prevents the degradation of the vitamin by the stomach acids and bacterial process in the small intestines.

The composition of the present invention is made by the following method. Most generally, the method includes forming nanoparticles of a vitamin, wherein the vitamin is surrounded by lipids for delivery through mucous membranes. More specifically, the vitamin (vitamin D3) is dissolved into MCT oil by sonication at 75 to 85 watts at a maximum of 10 KHz. The MCT oil/vitamin mixture is added to 3 liters of 18 Megaohm water and sonicated for 5 minutes at the above settings. Lecithin is added to the mixture and sonicated for 5 minutes at the above settings. Cholesterol is added to the above mixture and sonicated for 5 minutes at the above settings. Polysorbate 80 is added to the mixture and sonicated for 5 minutes at the above settings. The sonication is then adjusted to 600 watts at 15 KHz and the mixture is sonicated for a total of 15 minutes in 5-minute increments. The size of the material is checked to specifications of 35 to 45 nanometers including encapsulating material. FOODGARD® is added to the final mixture stirring in until dissolved. The resulting product is a water-based product that can be taken orally via spray or drops. These parameters are dependent on the material being nanosized and can vary for different vitamins.

The present invention also provides for a pharmaceutical composition, including nanoparticles encapsulating a vitamin, the nanoparticles including medium chain triglycerides, lecithin, cholesterol, and polysorbate 80 in a pharmaceutically acceptable carrier.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

The composition of the present invention is preferably formulated to be taken orally by mouth using a spray delivery method. However, other delivery methods can also be used. More generally, the composition is delivered to a mucous membrane in the body.

Therefore, the present invention provides for a method of increasing absorption of a vitamin, by administering a composition including nanoparticles of a vitamin surrounded by lipids in a pharmaceutically acceptable carrier to a mucous membrane in an individual, the composition penetrating the mucous membrane and providing faster transit to the liver than other delivery systems, thereby increasing absorption of the vitamin. Most preferably, the nanoparticles include the vitamin, medium chain triglycerides, lecithin, cholesterol, and polysorbate 80 as described above.

Upon delivery, an outer later of polysorbate 80 dissolves quickly to expose the cholesterol layer. This acts as a “lubricant” to enter the mucous membranes through the mouth. The oral mucosa is the mucous membrane lining the inside of the mouth and consists of stratified squamous epithelium termed oral epithelium and an underlying connective tissue termed lamina propria. Oral mucosa can be divided into three main categories based on function and histology: (1) Masticatory mucosa, keratinized stratified squamous epithelium, found on the dorsum of the tongue, hard palate and attached gingiva; (2) Lining mucosa, nonkeratinized stratified squamous epithelium, found almost everywhere else in the oral cavity, including buccal mucosa which refers to the inside lining of the cheeks and floor of the mouth, labial mucosa which refers to the inside lining of the lips, alveolar mucosa which refers to the lining between the buccal and labial mucosae, it is a brighter red, smooth and shiny with many blood vessels, and is not connected to underlying tissue by rete pegs; and (3) specialized mucosa, specifically in the regions of the taste buds on lingual papillae on the dorsal surface of the tongue that contains nerve endings for general sensory reception and taste perception.

The composition of the present invention is designed to penetrate and use these tissues as the gateway to the underlying capillary system. This allows a much faster transit time to the liver than other delivery systems so the vitamin D and be processed to its usable form. While the composition can penetrate all the tissues of the mouth, there is a preference for the lining mucosa areas of the mouth. This product can be absorbed with any mucosal tissue.

While the focus of the composition is utilizing the mucous membrane of the mouth, any mucous membrane described above can effectively transfer the composition. The function of the mucous membranes is to keep tissue moist (for example in the respiratory tract, including the mouth and nose). It also plays a role in absorbing and transforming nutrients. It is this absorbing trait of the mucosal membranes that the encapsulation of the vitamin D3 utilizes to transit to the capillary system for transport to the liver much more quickly then through the stomach then being absorbed by the mucosal tissues in the small intestines.

The invention is further described in detail by reference to the following experimental examples. These examples are provided for the purpose of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

Proof of Concept for Nanosized Vitamin Uptake Via Sublingual/Buccal Route

Purpose: to prove the hypothesis that the sublingual/buccal administration of a nanosized vitamin is biologically effective. Vitamin D3, the standard form of vitamin D supplementation was chosen because it is very poorly absorbed through the intestinal track. If it crosses, everything else should as well.

Ingredients

A. 1 g of pharmaceutical grade vitamin D3 was nanosized and encapsulated into a nano micelle using a proprietary process and dispersed into 2.91 L of water.

B. An over-the-counter soft gel capsule of Spring Valley brand D3 containing 400 IU of vitamin D3.

Subjects

Subject A is a 52-year-old Caucasian female, in apparent good health, taking no medications and no supplements for at least six months. Females normally have lower levels of vitamin D then do men.

Subject B is a Caucasian male of Norwegian extraction having immigrated to the US one year ago. He is healthy and taking no supplements or medications for at least six months. Scandinavians, for genetic reasons, absorb more vitamin D than do other genetic groups.

Methods

Subject A received 60 sprays over three hours of the Nano-sized vitamin D solution. Each spray had a volume of 0.6 ml for a total of 36 ml.

Subject B receive one capsule of Spring Valley vitamin D3 containing 400 IU swallowed with water.

Both subjects were given a couple of crackers with peanut butter and cheese to eat at the beginning of the experiment.

Blood was drawn by a trained phlebotomist at baseline and at one, two and three hours post dose.

The blood was tested at Access Medical labs using a high-power liquid chromatography, then mass spectrometer technique.

25D-OH D3 (a liver hydroxylation) and 25 hydroxyvitamin D were measured in all blood draws. 25 hydroxyvitamin D3 is the form of vitamin D after the first pass through the liver and 25 hydroxyvitamin D3 level is the best measure of full body stores of vitamin D. The normal lab value range is 30 ng/ML to 100 ng per ML.

Results

Subject A's baseline value was 17.1 ng per ML. Her values continued to rise over the testing period to a high at the 3 Hour draw of 20.5 ng per ML. This represents a three hour rise of 19.88%.

Subject B's baseline value was 28.0 ng per ML. His highest value was at the 2-hour sampling which was 31.9 ng per ML. This is equal to a 12.86% increase. However, the 3-hour draw showed a fall to 31.6 ng per ML.

At one-week post draw, subject A was tested again and her results where 44.2 ng per ML, an increase of over 100%. Blood will be drawn from both subjects at the two-week mark.

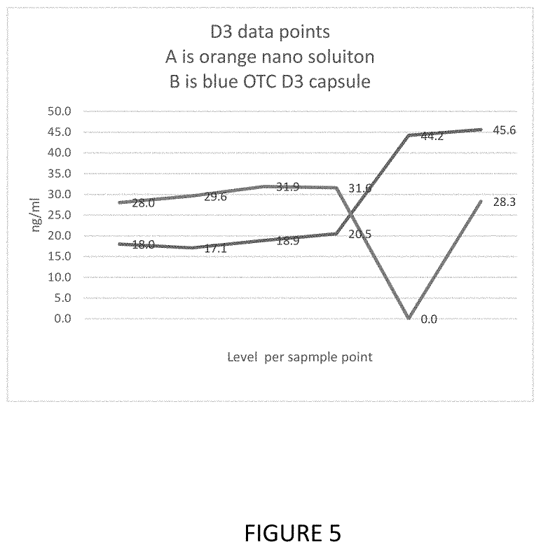

TABLE 2 shows data for subject A, and TABLE 3 shows data for subject B. FIG. 5 is a graph of the data.

TABLE 2
Subject A

| | Date | | | | | | |
|---|---|---|---|---|---|---|---|
| | 24 Oct. | 24 Oct. | 24 Oct. | 24 Oct. | 30 Oct. | 7 Nov. | 14 Nov. |
| ng/ml | 18 | 17.1 | 18.9 | 20.5 | 44.2 | 45.6 | 44.9 |

TABLE 3
Subject B

| | Date | | | | | |
|---|---|---|---|---|---|---|
| | 24 Oct. | 24 Oct. | 24 Oct. | 24 Oct. | 30 Oct. | 7 Nov. |
| ng/ml | 28 | 29.6 | 31.9 | 31.6 | | 28.3 |

### CONCLUSIONS

These results indicate that the sublingual administration of nano-sized vitamin D3 results in a rapid, almost immediate rise in blood levels. The hypothesis, thereby, was confirmed by this study. This encapsulation technology can be used to increase the effective absorption of any vitamin, both fat and water soluble.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A composition for increasing absorption and bioavailability of a vitamin, comprising:
nanoparticles encapsulating a vitamin, wherein said vitamin is surrounded by lipids for delivery through mucous membranes.

2. The composition of claim 1, wherein said vitamin is chosen from the group consisting of Vitamin A, Vitamin B₁, Vitamin B₂, Vitamin B₃, Vitamin B₅, Vitamin B₆, Vitamin B₁₂, Vitamin C, Vitamin D, Vitamin E, Vitamin K, and combinations thereof.

3. A composition for increasing absorption and bioavailability of a vitamin comprising:
nanoparticles encapsulating a vitamin, said nanoparticles including medium chain triglycerides (MCTs), lecithin, cholesterol, and polysorbate 80.

4. The composition of claim 3, wherein said vitamin is chosen from the group consisting of Vitamin A, Vitamin B₁, Vitamin B₂, Vitamin B₃, Vitamin B₅, Vitamin B₆, Vitamin B₁₂, Vitamin C, Vitamin D, Vitamin E, Vitamin K, and combinations thereof.

5. The composition of claim 3, wherein said MCTs are in an oil form.

6. The composition of claim 3, wherein said MCTs include C6, C8, C10, C12, and C14.

7. The composition of claim 3, wherein said composition further includes a natural antimicrobial.

8. The composition of claim 3, wherein said composition includes 2 g of vitamin D3, 4 g of medium chain triglycerides oil, 0.51688 g of lecithin, 0.0256 g of cholesterol, 1.4332 g of polysorbate 80, and 12 g of a natural antimicrobial.

9. The composition of claim 3, wherein said vitamin is surrounded by lipids for delivery through mucous membranes.

10. The composition of claim 3, wherein said nanoparticles are 35-45 nm in size.

11. A pharmaceutical composition for increasing absorption and bioavailability of a vitamin, comprising:
nanoparticles encapsulating a vitamin, said nanoparticles including medium chain triglycerides (MCTs), lecithin, cholesterol, and polysorbate 80, in a pharmaceutically acceptable carrier.

12. The pharmaceutical composition of claim 11, wherein said vitamin is chosen from the group consisting of Vitamin A, Vitamin B₁, Vitamin B₂, Vitamin B₃, Vitamin B₅, Vitamin B₆, Vitamin B₁₂, Vitamin C, Vitamin D, Vitamin E, Vitamin K, and combinations thereof.

13. The pharmaceutical composition of claim 11, wherein said MCTs are in an oil form.

14. The pharmaceutical composition of claim 11, wherein said MCTs include C6, C8, C10, C12, and C14.

15. The pharmaceutical composition of claim 11, wherein said composition further includes a natural antimicrobial.

16. The pharmaceutical composition of claim 11, wherein said composition includes 2 g of vitamin D3, 4 g of medium chain triglycerides oil, 0.51688 g of lecithin, 0.0256 g of cholesterol, 1.4332 g of polysorbate 80, and 12 g of a natural antimicrobial.

17. The pharmaceutical composition of claim 11, wherein said pharmaceutical composition is in a form chosen from the group consisting of a spray and drops.

18. A method of making a composition for increasing absorption and bioavailability of a vitamin, including the steps of:
forming nanoparticles encapsulating a vitamin, wherein the vitamin is surrounded by lipids for delivery through mucous membranes.

19. The method of claim 18, wherein said forming step is further defined as dissolving the vitamin into medium chain triglyceride (MCT) oil by sonicating to form an MCT oil/vitamin mixture, adding the MCT oil/vitamin mixture to 3 liters of 18 Megaohm water and sonicating for 5 minutes, adding lecithin and sonicating for 5 minutes, adding cholesterol and sonicating for 5 minutes, adding polysorbate 80 and sonicating for 5 minutes, and sonicating at 600 watts at 15 KHz for 15 minutes in 5-minute increments.

20. The method of claim 19, further including the step of adding a natural antimicrobial by stirring until dissolved.

21. The method of claim 18, wherein said method results in a water-based composition.

22. The method of claim 18, wherein the vitamin is chosen from the group consisting of Vitamin A, Vitamin B₁, Vitamin B₂, Vitamin B₃, Vitamin B₅, Vitamin B₆, Vitamin B₁₂, Vitamin C, Vitamin D, Vitamin E, Vitamin K, and combinations thereof.

23. A method of increasing absorption of a vitamin, including the steps of:
administering a composition including nanoparticles encapsulating a vitamin, wherein the vitamin is surrounded by lipids in a pharmaceutically acceptable carrier to a mucous membrane in an individual;
the composition penetrating the mucous membrane and providing faster transit to the liver than other delivery systems; and
increasing absorption of the vitamin.

24. The method of claim 23, wherein the composition is further defined as including nanoparticles of a vitamin, medium chain triglycerides (MCTs), lecithin, cholesterol, and polysorbate 80.

25. The method of claim 24, wherein said penetrating step is further defined as lipids in the composition allowing mucosa cells to part and gaining access to capillary systems in the individual.

26. The method of claim 25, wherein said penetrating step is further defined as dissolving an outer layer of polysorbate 80 and exposing a cholesterol layer of the composition, wherein the cholesterol layer acts as a lubricant to enter mucosal tissue in the individual.

27. The method of claim 26, wherein the mucosal tissue is in the mouth and is chosen from the group consisting of masticatory mucosa, lining mucosa, specialized mucosa, and combinations thereof.

28. The method of claim 26, wherein the mucosal tissue is chosen from the group consisting of bronchial mucosa and the lining of vocal folds, mucosa of the uterus, esophageal mucosa, gastric mucosa, intestinal mucosa, nasal mucosa, olfactory mucosa, oral mucosa, penile mucosa, vaginal mucosa, frenulum of tongue, tongue, anal canal, and palpebral conjunctiva.

29. The method of claim 23, further including the step of preventing degradation of the vitamin by stomach acids and small intestine bacteria.
